(19) United States
(12) Patent Application Publication
(10) Pub. No.: US 2008/0318912 A1
(43) Pub. Date: Dec. 25, 2008

(54) Medicaments for Treating Chronic Respiratory Disease

(76) Inventors: Craig Fox, Harlow (GB); Harry Finch, Harlow (GB); Mary Fitzgerald, Harlow (GB)

Correspondence Address: MORGAN LEWIS & BOCKIUS LLP, 1111 PENNSYLVANIA AVENUE NW, WASHINGTON, DC, 20004, US

(21) Appl. No.: 11/659,458

(22) PCT Filed: Aug. 3, 2005
(86) PCT No.: PCT/GB2005/003039

§ 371 Date: Apr. 18, 2008

(30) Foreign Application Priority Data
Aug. 5, 2004 (GB) 0417481.9

(51) Int. Cl. A61K 31/58; A61P 11/00

(52) U.S. Cl. 514/171

(57) ABSTRACT

There is provided the use of a methylxanthine compound and a steroid in a synergistic combination for the treatment of a respiratory disease, wherein the methylxanthine compound is administered at a dose, which, in isolation, is not effective to treat said disease.

The present invention provides the use of methylxanthine derivatives such as theophylline and steroid drugs in a synergistic combination for the treatment of chronic obstructive pulmonary disease (COPD). The administration of a steroid and theophylline in combination, at doses where each individual component has no, or minimal, anti-inflammatory effect, results in a therapeutic synergistic anti-inflammatory response.

### INTRODUCTION

Theophylline is an inexpensive white crystalline powder used as an oral agent for chronic respiratory diseases such as asthma and COPD. Aminophylline, or theophylline ethylenediamine, is a combination of theophylline and ethylenediamine and has similar properties. Theophylline is known to have a bronchodilating effect and a mild anti-inflammatory effect, due in part to its activity as a weak nonselective phosphodiesterase (PDE) inhibitor. The drug has hitherto been characterised by a narrow therapeutic index, and toxicity to this agent, marked by gastrointestinal upset, tremor, cardiac arrhythmias, and other complications, is common in clinical practice. Other drugs for chronic respiratory diseases, such as inhaled beta-agonists and inhaled steroids, are often prescribed instead of theophylline to avoid its adverse effects.

Although theophylline has been in clinical use for many years, its molecular mechanism of action and its site of action remain uncertain. Several molecular mechanisms of action have been proposed, including the following.

Theophylline is a weak and nonselective inhibitor of phosphodiesterases, which break down cyclic nucleotides in the cell, thereby leading to an increase in intracellular cyclic AMP and GMP concentrations. Theophylline relaxes airway smooth muscle by inhibition of PDE activity (PDE3, PDE4 and PDE5), but relatively high concentrations are needed for maximal relaxation (Rabe, et al. Eur Respir J 1999, 8: 637-42). The degree of PDE inhibition is very small at concentrations of theophylline that are therapeutically relevant. There is no evidence that theophylline has any selectivity for any particular isoenzyme, such as, for example, PDE4B, the predominant PDE isoenzyme in inflammatory cells that mediates anti-inflammatory effects in the airways.

Theophylline is a potent inhibitor of adenosine receptors at therapeutic concentrations, with antagonism of A₁ and A₂ receptors, although it is less effective against A₃ receptors (Pauwels, R. A., Joos, G. F. Arch Int Pharmacodyn Ther 1995, 329: 151-60).

Theophylline increases interleukin-10 release, which has a broad spectrum of anti-inflammatory effects. This effect may be mediated via PDE inhibition, although this has not been seen at the doses that are effective in asthma (Oliver, et al. Allergy 2001, 56: 1087-90).

Theophylline prevents the translocation of the proinflammatory transcription factor nuclear factor-κB (NF-κB) into the nucleus, thus potentially reducing the expression of inflammatory genes in asthma and COPD (Tomita, et al. Arch Pharmacol 1999, 359: 249-55). These effects are seen at high concentrations and may also be mediated by inhibition of PDE.

Theophylline has moreover recently been shown to activate histone deacetylase (HDAC). Acetylation of histone proteins is associated with activation of gene function, and it is believed that proinflammatory transcription factors which activate inflammatory genes also cause an increase in histone actetyltransferase activity. By increasing HDAC activity and so deacetylating histone proteins, theophylline is believed to suppress the expression of inflammatory genes (see Barnes, (2003) Am J Respir Crit. Care Med 167:813-818).

Glucocorticoid drugs (steroids) have become the therapy of choice in asthma and are widely used in the treatment of COPD, usually in inhaled form. However, although inhaled steroids are effective in the majority of asthma patients their use in COPD is contentious owing to their lack of demonstrable anti-inflammatory effect (Culpitt, S. V. et al. (1999). Am. J. Respir. Crit. Care Med. 160, 5 Pt 1, 1635-1639) and their apparent failure to affect disease progression (Burge, et al (2000). BMJ 320: 1297-1303). Asthma patients who fail to respond to low doses of steroids are administered a higher dose, in the case of budesonide up to 1600 μg daily.

Evans et al., (2004) NEJM 337:1412, suggest that high doses of inhaled steroids may be substituted by administration of a normal glucocorticoid dose, together with a low dose of theophylline for use in asthma. Patients were administered 400 μg of budesonide (the standard dose) together with 250 or 375 mg of theophylline, or 800 μg of budesonide plus placebo, twice daily. The plasma concentrations of theophylline that were achieved in this study ranged from 2.5 to 17.1 mg/l with a median value of 8.7 mg/l. The effects of these two treatment paradigms were similar suggesting that theophylline has dose sparing effects when given with a steroid. However, at the doses used, patients suffered from drug-related side effects, including gastrointestinal upsets, palpitations, sore throats and other side-effects associated with steroids and/or theophylline therapy. Moreover, the authors did not determine any effects of the drugs on inflammation. Similar studies investigating the potential interaction between inhaled steroids and oral theophylline have not been carried out in COPD patients

There is thus a need for a therapeutic regime for COPD which provides effective anti-inflammatory activity and avoids side-effects associated with existing therapies.

### BRIEF DESCRIPTION OF THE INVENTION

The present inventors have determined that steroids and methylxanthine compounds, administered at doses which alone are not effective in treating inflammation induced by tobacco smoke (TS) in an animal model of COPD, when administered together have a synergistic effect and are able to markedly reduce inflammation in said models, by 50% or more in the tests set forth below. TS exposure is widely accepted to be the principal cause of COPD in human beings.

In a first aspect, therefore, there is provided the use of a methylxanthine compound and a steroid for combined use in the manufacture of a composition for the treatment of a chronic respiratory disease, wherein the methylxanthine compound is administered at a dose which, in isolation, is not effective in treating said respiratory disease, but together with the steroid is effective in reducing inflammation in the respiratory tract.

Preferably, the chronic disease is COPD. Advantageously, the chronic disease may include severe asthma and cystic fibrosis.

The invention recognises a synergistic activity between a methylxanthine compound and steroid drugs which results in an extremely high anti-inflammatory activity. This synergy is achieved using doses of the drugs which were ineffective when administered alone. The effect is not additive, but synergistic, in that two drugs having little or no effect can be administered simultaneously to obtain highly significant inhibition of the inflammatory response.

A methylxanthine compound, as used herein, refers to theophylline and pharmacologically equivalent compounds and salts, including aminophylline and oxtriphylline. Such compounds are methylxanthines, which includes caffeine, Theobromine, Furaphylline, 7-propyl-theophylline-dopamine, enprofylline, and the like. Steroid drugs include glucocorticoids, corticosteroids and mineralocorticoids, such as dexamethasone and budesonide, beclomethasone, flunisolide, fluticasone, Ciclesonide, mometasone, hydrocortisone, prednisone, prednisolone, triamcinolone, betamethasone, fludrocoritisone and desoxycorticosterone. Steroid drugs can additionally include steroids in clinical development for COPD such as GW-685698, GW-799943 and compounds referred to in international patent applications WO0212265, WO0212266, WO02100879, WO03062259, WO03048181 and WO03042229. Steroid drugs can additionally include next generation molecules in development with reduced side effect profiles such as selective glucocorticoid receptor agonists (SEGRAs), including ZK-216348 and compounds referred to in international patent applications WO00032585, WO000210143, WO2005034939, WO2005003098, WO2005035518 and WO2005035502.

Preferably, the methylxanthine is theophylline.

In accordance with the invention, the steroid may be administered at a standard dose, or a dose which would have no effect if administered independently of the methylxanthine compound to an individual.

Advantageously, the steroid is ineffective in reducing inflammation in said respiratory disease at the dose used. Certain respiratory diseases, including COPD, are resistant to steroid treatment and steroid drugs are ineffective in reducing inflammation. Together with theophylline, however, an anti-inflammatory effect is observed.

Administration may take place by any appropriate route, including orally, by inhalation, by injection, by means of long-term releasing implants, and the like. Oral administration is advantageous, especially in underdeveloped countries where the handling of injectables is problematic, and in over-the-counter medical applications. Inhaled medications are of course familiar to sufferers of chronic respiratory diseases such as asthma, where inhalers are in common use. Preferably, the theophylline is administered orally.

In another aspect, the invention provides a pharmaceutical composition in unit dosage form, comprising a methylxanthine compound at a dose which is insufficient to be effective in the treatment of a respiratory disease if administered independently, and a steroid. Such unit dosages may be packaged to provide a kit for the treatment of respiratory disease, comprising a methylxanthine compound and a steroid in unit dosage form, wherein the methylxanthine compound is at a dose which is insufficient to be effective in the treatment of a respiratory disease if administered independently.

Such a kit may comprise, for example, instructions for use which direct the user to administer the medicaments substantially simultaneously, such that they are present in the patient's body at the same time.

The invention further provides a methylxanthine compound and a steroid in unit dosage form, wherein the methylxanthine compound is at a dose which is insufficient to be effective in the treatment of a respiratory disease if administered independently, for simultaneous, simultaneous separate or sequential use in the treatment of respiratory disease.

In the kits or unit dosages according to the invention, the steroid is preferably present at a dose which is insufficient to be effective in the treatment of a respiratory disease if administered independently.

The invention further provides a methylxanthine compound and a steroid in unit dosage form, wherein the methylxanthine compound is provided at a dose which is insufficient to be effective in the treatment of a respiratory disease if administered independently, for simultaneous, simultaneous separate or sequential use in the treatment of a respiratory disease.

In the foregoing aspects of the invention, the oral dosage of the methylxanthine compound which does not exert any therapeutic or pharmacological effect is advantageously below 5 mg/kg, preferably between 0.1 and 4 mg/kg, most preferably between 0.1 and 3 mg/kg. Advantageously, the dose of methylxantine is 3 mg/kg or less. Plasma levels achieved with these doses of methylxanthine fall below those currently considered necessary for clinical efficacy (10-20 mg/l) (Cazzola et al., (2004) Pulmonary Pharmacology & Therapeutics 17, 141-145).

In the foregoing aspects of the invention, the dosage of steroid which does not exert any apparent pharmacological effect in the animal model of COPD is advantageously below 0.5 mg/kg, preferably between 0.1 and 0.4 mg/kg, most preferably between 0.1 and 0.3 mg/kg. Advantageously, the dose of steroid is 0.3 mg/kg or less.

The effectiveness of the treatment may be assayed, in accordance with the invention, by any technique capable of assessing inflammation. In a preferred embodiment, the treatment of the respiratory disease is assessed by counting cells recovered by bronchoalveolar lavage (BAL). Inflammation can also be assessed in sputum or in bronchial epithelial biopsies.

Advantageously, the cells are selected from the group consisting of macrophages, epithelial cells, neutrophils, eosinophils and lymphocytes.

The invention is capable of substantially reducing inflammation in respiratory diseases. Advantageously the cell count is reduced by 50% or more upon administration of a methylxanthine compound and a steroid, preferably 70% or more.

At the same time, the individual doses of a methylxanthine compound and the steroid can advantageously reduce cell numbers by a total, when added together, of 40% or less, preferably 30% or less, and ideally by 20% or less. Where the synergistic reduction of cell count on administration of a methylxanthine compound and a steroid is 70% or more, the additive effect of the individual agents is preferably 60% or less, advantageously 56% or less.

### BRIEF DESCRIPTION OF THE FIGURES

FIG. 1 Effect of theophylline and dexamethasone given orally (1 h prior to and 6 h post 11 consecutive daily exposures to TS) either alone or in combination on total cell numbers recovered in the BAL 24 h post final exposure. Theophylline was given alone at 3 mg/kg or in combination with Dexamethasone (0.3 mg/kg) at 3 and 1 mg/kg.

FIG. 2 Effect of theophylline and dexamethasone given orally (1 h prior to and 6 h post II consecutive daily exposures to TS) either alone or in combination on macrophage numbers recovered in the BAL 24 h post final exposure. Theophylline was given alone at 3 mg/kg or in combination with Dexamethasone (0.3 mg/kg) at 3 and 1 mg/kg.

FIG. 3 Effect of theophylline and dexamethasone given orally (1 h prior to and 6 h post 11 consecutive daily exposures to TS) either alone or in combination on epithelial cell numbers recovered in the BAL 24 h post final exposure. Theophylline was given alone at 3 mg/kg or in combination with Dexamethasone (0.3 mg/kg) at 3 and 1 mg/kg.

FIG. 4 Effect of theophylline and dexamethasone given orally (1 h prior to and 6 h post 11 consecutive daily exposures to TS) either alone or in combination on neutrophil numbers recovered in the BAL 24 h post final exposure. Theophylline was given alone at 3 mg/kg or in combination with Dexamethasone (0.3 mg/kg) at 3 and 1 mg/kg.

FIG. 5 Effect of a theophylline and dexamethasone given orally (1 h prior to and 6 h post LPS) on LPS induced increases in total BAL cells 24 h post challenge.

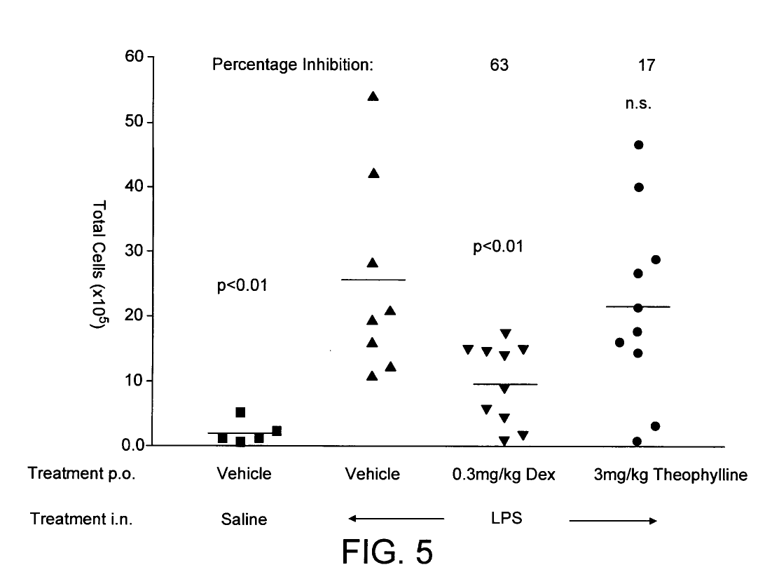

FIG. 6 Effect of a theophylline and dexamethasone given orally (20 and 1 h prior to and 6 h post LPS) on LPS induced increases in total BAL neutrophils 24 h post challenge.

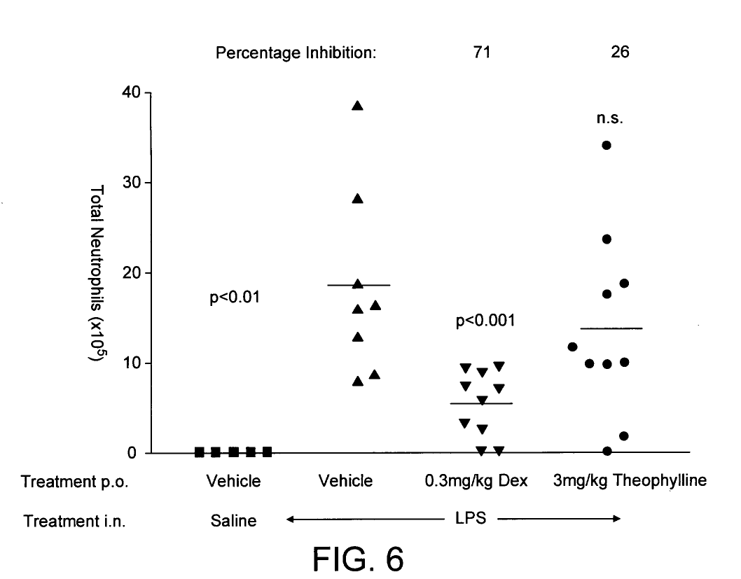

FIG. 7 Plasma concentrations on theophylline after oral dosing in A/J mice.

### DETAILED DESCRIPTION OF THE INVENTION

The present invention employs standard techniques of pharmacology and biochemistry, as described in more detail below. In the context of the invention, certain terms have specific meanings, as follows.

The invention describes the administration of methylxanthine and steroid drugs in combination, and contrasts the combined administration with individual administration of said drugs in isolation. “In isolation” accordingly refers to the administration of a methylxanthine compound without a steroid, or vice versa, irrespective of whether the steroid is administered before, concomitantly with or after the methylxanthine compound. The intention is to differentiate between the methylxanthine compound and the steroid being administered such that they can exert their pharmacological activities in the target organism contemporaneously or separately.

“Combined use” or “combination” within the meaning of the present invention is to be understood as meaning that the individual components can be administered simultaneously (in the form of a combination medicament), separately but substantially simultaneously (for example in separate doses) or sequentially (directly in succession or after a suitable time interval, provided that both agents are active in the subject at the same time).

“Effective”, referring to treatment of inflammatory conditions and/or respiratory disease, refers to obtaining a response in an assay which measures inflammation in respiratory disease. The preferred assay is bronchoalveolar lavage (BAL) followed by cell counting, wherein then presence of cells indicates inflammation of the lung. In human patients, BAL, induced sputum and bronchial biopsy are preferred methods of assessing inflammation. Inflammation may be induced by any desired means, such as tobacco smoke inhalation, administration of irritants such as LPS, and the like. Tobacco smoke inhalation is preferred since, as shown herein, the use of LPS does not faithfully reproduce an inflammatory response that is steroid resistant as is seen in COPD. In the context of the BAL/cell counting assay, “effective” preferably encompasses a reduction in cell numbers by 30%, 35%, 40%, 45%, 50%, 60%, 65%, 70% or more compared to a control in which the agent is not administered.

“Not effective” means, in the same assay, a much lower level of response. Preferably, in the BAL/cell counting assay, “not effective” means that the reduction in cell numbers is 30% or below, advantageously 28%, 27%, 26%, 25%, 24%, 23% 22% or 21% or below, and preferably 20% or below. In some instances, “not effective” can encompass an increase in inflammation, seen for example as an increase in cell numbers.

“Synergistic” means that the effectiveness of two agents is more than would be expected by summing their respective individual effectiveness in a given assay. For example, if a methylxanthine compound and a steroid reduce cell numbers in the BAL assay by 10% and 20% respectively when administered in isolation, a synergistic response would be seen if the reduction in cell numbers were above 30% in a combined administration of the same agents at the same dose.

“Administered” refers to the administration of the entire dose of the agent, such as in a bolus dose, to the intended subject. In the context of the present invention, dosage is preferably expressed in terms of plasma levels achieved (<5 mg/l; 6-9 mg/l; 10-20 mg/l) with plasma levels preferably between 1 to 9 mg/l, and most preferably less than 1 mg/l.

A “dose” is an amount of agent administered as described above. Administration may be by any suitable route, including the routes referred to above. In general, it is not possible to equate dosages given by two routes of administration; for example, inhaled steroids generally are administrable at lower doses than oral steroids to achieve a comparative effect, since they are delivered directly to the site of action rather than systemically.

“Unit dosage” form, is a preparation of a pharmaceutical composition in one or more packaged amounts, each of which contains a single dosage in accordance with the invention. Typical unit dosages include pills, capsules, suppositories, single-use ampoules and the like.

### Theophylline and Steroids

### Theophylline and Amminophylline

Theophylline has the structure shown below:

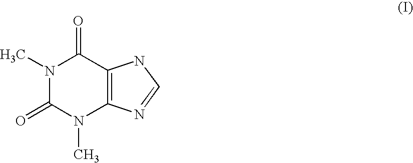

and is available commercially under a variety of brand anmes, including Accurbron, Aerobin, Aerolate, Afonilum, Aquaphyllin, Armophylline, Asmalix, Austyn, Bilordyl, Bronchoretard, Bronkodyl, Cetraphylline, Constant T, Duraphyllin, Diffumal, Elixomin, Elixophyllin, Etheophyl, Euphyllin, Euphylong, LaBID, Lanophyllin, Lasma, Nuelin, Physpan, Pro-Vent, PulmiDur, Pulmo-Timelets, Quibron, Respid, Sio-Bid, Slo-Phyllin, Solosin, Sustaire, Talotren, Teosona, Theobid, Theoclear, Theochron, Theo-Dur, Theolair, Theon, Theophyl, Theograd, Theo-Sav, Theospan, Theostat, Theovent, T-Phyl, Unifyl, Uniphyl, Uniphyllin, and Xanthium. The chemical name of Theophylline is 3,7-dihydro-1,3-dimethyl-1H-purine-2,6-dione or 1,3-Dimethylxanthine and its general chemical formula is C₇H₈N₄O₂.

### Aminophylline

A theophylline derivative, this is synonymous with theophylline ethylenediamine. Aminophylline is a derivative of theophylline, both are methylxanthines and are derived from Xanthines. The drug aminophylline differs somewhat in its stricture from theophylline in that it contains ethylenediamine, as well as more molecules of water. Aminophylline tends to be less potent and shorter acting than theophylline. Its structure is shown below:

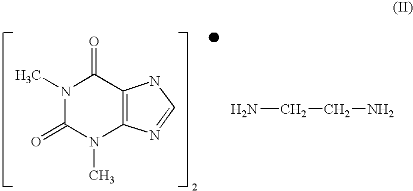

Theophylline is well absorbed from the gastrointestinal tract with up to 90-100 percent bioavailability. Peak levels are achieved within 1-2 hours following ingestion, but this is slowed by the presence of food. Theophylline is approximately 60 percent plasma protein bound and has a mean volume of distribution of 0.51/kg. Plasma protein binding is reduced in infants and in patients with liver cirrhosis. The mean plasma half-life of theophylline is about 8 hours in adults although there is large intra- and interindividual variation, and also varies greatly with age being approximately 30 hours in premature neonates, 12 hours within the first 6 months, 5 hours up to the first year of life and approximately 3.5 hours up to the age of 20 gradually increasing again thereafter. Because of the relatively short plasma half-life of theophylline, there are many sustained release preparations available commercially. These all vary as to their bioavailability and the time to peak plasma concentrations (see further below).

Theophylline is mainly metabolised in the liver by demethylation or oxidation using the cytochrome P450 system. Only small amounts are excreted by the kidney unchanged, and dosage adjustments in renal failure are unnecessary. However, caution needs to be exercised when using other drugs that are also metabolised by the cytochrome system when dosage adjustments need to be made in conjunction with the measurement of plasma levels. Many drugs may interfere with the metabolism of theophylline. Special care should be taken with certain antibiotics as patients with acute infective exacerbations of their airways obstruction may be inadvertently put on them without consideration of the effects on theophylline metabolism. These include the macrolide (e.g. erythromycin) and quinolone (e.g. ciprofloxacin) families of antibiotics which both reduce theophylline clearance to varying degrees. Other drugs that reduce theophylline clearance include cimetidine, allopurinol and propanolol (although this would be a rather unusual therapeutic combination). Drugs that increase theophylline metabolism include rifampicin, phenobarbitone and particularly phenyloin and carbamazepine but not the oral contraceptive pill. The rate of metabolism of theophylline is increased substantially in cigarette smokers (the half life can be halved), although may not be significant in those who smoke less than 10/day. Smoking marijuana has a similar effect as can eating a high protein diet. Hepatic dysfunction, heart failure and cor pulmonale all reduce the elimination of theophylline, and low albumin states reduce the amount of protein bound drug in the blood, so results of plasma levels need to be interpreted with caution. Therefore, as the clinical state of the patient with heart failure or respiratory failure with cor pulmonale improves, the clearance of theophylline alters, and dosage adjustments may be necessary.

### Methylxanthines

Methylxanthine compounds, which include throphylline and aminophlylline, have the general formula

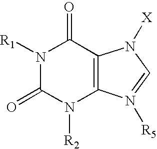

### Wherein

X represents hydrogen, an aliphatic hydrocarbon radical or —CO—NR₃R₄,
R₁, R₂ and R₃ represent aliphatic hydrocarbon radicals;
R₄ represents hydrogen or an aliphatic hydrocarbon radical and R₃ and R₄ together with the nitrogen atom may also represent an alkylene imino radical with 5 to 6 ring members or the morpholino radical; and
R₅ represents hydrogen or an aliphatic hydrocarbon radical.

All such compounds are within the scope of the present invention; however, theophylline itself is especially preferred.

### Steroids

Steroid drugs in general are suitable for use in the present invention. Particular steroids are set forth below.

### Common Inhaled Steroids Include:

Pulmicort® (budesonide)
Flovent® (fluticasone)
Asmanex® (mometasone)
Alvesco® (cilcesonide)
Aerobid® (flunisolide)
Azmacort® (triamcinolone)
Qvar® (beclomethasone HFA)
Steroids may also be administered in the form of combinations with long acting bronchodilators with a range of mechanisms including beta 2 adrenergic agonists and/or muscarinic antagonists. The bronchodilator included in the steroid combination can have beta 2 adrenergic agonist and muscarinic antagonist activity in the same molecule.
Advair® (Flovent® and Serevent®) Note: Serevent® is the long acting beta-agonist salmeterol.
Symbicort® (Pulmicort® and Oxis®) Note: Oxis is the long acting beta-agonist formoterol.

### Common Steroid Pills and Syrups Include:

Deltasone® (prednisone)
Medrol® (methylprednisolone)
Orapred®, Prelone®, Pediapred® (prednisolone)

### Budesonide

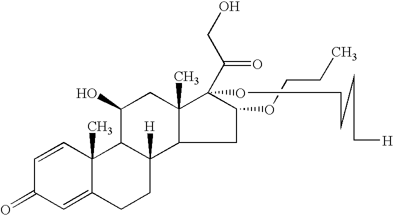

Chemical name: C₂₅H₃₄O₆: 430.54 (+)-[(RS)-16a, 17a-Butylidenedioxy-11b, 21-dihydroxy-1,4-pregnadiene-3,20-dione]

CAS Registry Number: 51333-22-3

Budesonide was originally synthesised from 16α-hydroxyprednisolone. The unique structure of the molecule is the key to its combination of high topical anti-inflammatory potency with relatively low potential for systemic side-effects. In addition, budesonide is both sufficiently water soluble for easy dissolution in mucosal fluids and lipid soluble for rapid uptake by mucosal membranes. Because the acetal group is asymmetrical, budesonide exists as a 1:1 mixture of two epimers, known as 22R and 22S.

### Fluticasone

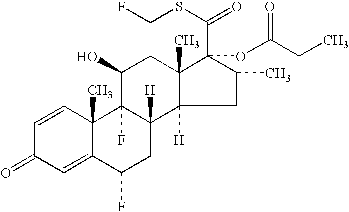

BRAND_NAMES: Cutivate, Flixonase, Flixotide, Flonase, Flovent, Flunase

### CHEMICAL_NAME:

(6(,11 (,16(,17( )-6,9-difluoro-11-hydroxy-16-methyl-3-oxo-17-(1-oxopropoxy)androsta-1,4-diene-17-carbothioic acid S-(fluoromethyl)ester

CHEMICAL_FORMULA: C₂₅H₃₁F₃O₅S

### CAS_NUMBER: 80474-14-2

### Beclomethasone

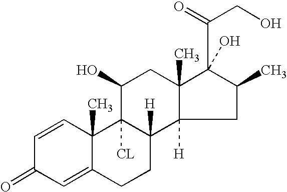

### CHEMICAL_NAME

(11(,16( )-9-chloro-11,17,21-trihydroxy-16-methylpregna-1,4-diene-3,20-dione

### CHEMICAL_FORMULA

C₂₂H₂9ClO₅

### CAS_NUMBER

4419-39-0

### BRAND NAMES (VARIANT)

Aerobec (beclomethasone dipropionate), Aldecin (beclomethasone dipropionate), Anceron (beclomethasone dipropionate), Andion (beclomethasone dipropionate), Beclacin (beclomethasone dipropionate), Becloforte (beclomethasone dipropionate), Beclomet (beclomethasone dipropionate), Beclorhinol (beclomethasone dipropionate), Becloval (beclomethasone dipropionate), Beclovent (beclomethasone dipropionate), Becodisks (beclomethasone dipropionate), Beconase (beclomethasone dipropionate), Beconasol (beclomethasone dipropionate), Becotide (beclomethasone dipropionate), Clenil-A (beclomethasone dipropionate), Entyderma (beclomethasone dipropionate), Inalone (beclomethasone dipropionate), Korbutone (beclomethasone dipropionate), Propademm (beclomethasone dipropionate), Qvar (beclomethasone dipropionate), Rino-Clenil (beclomethasone dipropionate), Sanasthmax (beclomethasone dipropionate), Sanasthmyl (beclomethasone dipropionate), Vancenase (beclomethasone dipropionate), Vanceril (beclomethasone dipropionate), Viarex (beclomethasone dipropionate), and Viarox (beclomethasone dipropionate).

### Triamcinolone

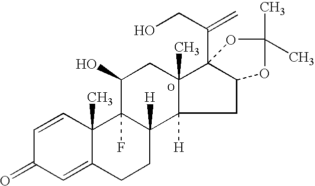

### BRAND_NAMES

Aristocort, Aristospan, Azmacort, Kenalog Nasacort

### CHEMICAL_NAME

(11 (,16( )-9-fluoro-11,21-dihydroxy-16,17-[1-methylethylidenebis(oxy)]pregna-1,4-diene-3,20-dione

### CHEMICAL_FORMULA

C₂₄H₃₁FO₆

### CAS_NUMBER

76-25-5

### Salmeterol/Advair

### BRAND_NAMES

*1-hydroxy-2-naphthoate*1-hydroxy-2-naphthoate: Arial, Salmetedur, Serevent

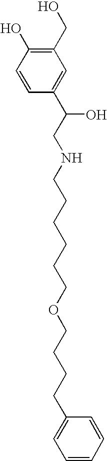

### CHEMICAL_NAME

(( )-4-hydroxy-(′-[[[6-(4-phenylbutoxy)hexyl]amino]methyl]-1,3-benzenedimethanol

### CHEMICAL_FORMULA

C₂₅H₃₇NO₄

### CAS_NUMBER

89365-50-4

### Methylprednisolone

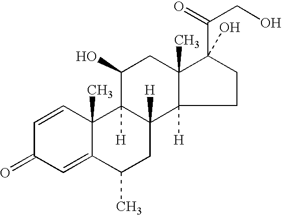

### CHEMICAL_NAME

(6(11( )-11,17,21-trihydroxy-6-methylpregna-1,4-diene-3,20-dione

### CHEMICAL_FORMULA

C₂₂H₃₀O₅

### CAS_NUMBER

83-43-2

### BRAND_NAMES

Medrate, Medrol, Medrone, Metastab, Metrisone, Promacortine, Suprametil, Urbason

### Prednisone

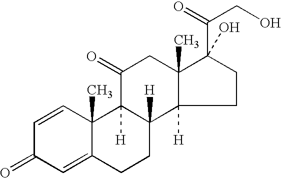

### BRAND_NAMES

Ancortone, Colisone, Cortancyl, Dacortin, Decortancyl, Decortin, Delcortin, Deltacortone, Deltasone, Deltison, Di-Adreson, Encorton, Meticorten, Nurison, Orasone, Paracort, Prednilonga, Pronison, Rectodelt, Sone, Ultracorten

### CHEMICAL_NAME

17,21-dihydroxypregna-1,4-diene-3,11,20,trione

### CHEMICAL_FORMULA

C₂₁H₂₆O₅

### CAS_NUMBER

53-03-2

### Formulation

Xanthine derivatives such as theophylline and aminophylline are widely available in a variety of pharmaceutical preparations including sustained release, transdermal delivery formulations, preparations for oral or inhaled (nasal) delivery. Likewise, steroid drugs are widely available in a variety of formulations. Formulations used in the examples described herein are further detailed below, but any formulation may be used in the present invention which allows delivery of the drug to the subject in the desired dosage.

In general, the pharmaceutical preparation may be one which can be given orally, intravenously, per inhalation, per rectum or transdermally.

Preferred compositions for use according to the invention may suitably take the form of tablets, capsules, granules, spheroids, powders or liquid preparations.

Tablets and capsules for oral administration may be prepared by conventional techniques with pharmaceutically acceptable excipients such as binding agents, fillers, lubricants, disintegrants, wetting agents, colourants and flavours. The tablets may be coated according to methods well known in the art.

Preferably the compositions produced or used in accordance with the invention is in dosage unit form, e.g. in tablet or filled capsule form. Further, it is envisaged that the active substance be in controlled release form.

Suitable materials for inclusion in a controlled release matrix include, for example:

(a) Hydrophilic or hydrophobic polymers, such as gums, cellulose esters, cellulose ethers, protein derived materials, nylon, acrylic resins, polyactic acid, polyvinylchloride, starches, polyvinylpyrrolidones, cellulose acetate phthalate. Of these polymers, cellulose ethers especially substituted cellulose ethers such as alkylcelluloses (such as ethylcellulose), C₁₋₆ hydroalkylcelluloses (such as hydroxypropylcellulose and especially hydroxyethyl cellulose) and acrylic resins (for example methacrylates such as methacrylic acid copolymers) are preferred. The controlled release matrix may conveniently contain between 1% and 80% (by weight) of the hydrophilic or hydrophobic polymer.

(b) Digestible, long chain (C₈-C₅₀, especially C₈-C₄₀), substituted or unsubstituted hydrocarbons, such as fatty acids, hydrogenated vegetable oils, such as Cutina™, fatty alcohols (such as lauryl, myristyl, stearyl, cetyl or preferably cetostearyl alcohol), glyceryl esters of fatty acids for example glyceryl monostearate mineral oils and waxes (such as beeswax, glycowax, caster wax or carnauba wax). Hydrocarbons having a melting point of between 20° C. and 90° C. are preferred. Of these long chain hydrocarbon materials, fatty (aliphatic) alcohols are preferred. The matrix may contain up to 60% (by weight) of at least one digestible, long chain hydrocarbon.

(c) Polyalkylene glycols. The matrix may contain up to 60% (by weight) of at least one polyalkylene glycol.

The medicament-containing controlled release matrix can readily be prepared by dispersing the active ingredient in the controlled release system using conventional pharmaceutical techniques such as wet granulation, dry blending, dry granulation or coprecipitation.

The agents of the invention may be administered in inhaled form. Aerosol generation can be carried out, for example, by pressure-driven jet atomizers or ultrasonic atomizers, but advantageously by propellant-driven metered aerosols or propellant-free administration of micronized active compounds from inhalation capsules.

The active compounds are dosed as described depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparata are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described in European Patent Application EP 0 505 321).

Respiratory diseases treated by the present invention include in particular allergen- and inflammation-induced bronchial disorders (bronchitis, obstructive bronchitis, spastic bronchitis, allergic bronchitis, allergic asthma, bronchial asthma, Cystic Fibrosis and COPD), which can be treated by the combination according to the invention. The synergistic combination of the invention is particularly indicated in long-term therapy, since lower quantities of drugs are needed than in conventional monotherapies.

### 1. Materials

Compounds were purchased from an external supplier. Carboxymethyl-cellulose (CMC) (Na salt) (product code C-4888) was obtained from Sigma. Phosphate buffered saline (PBS) was obtained from Gibco. Sterile saline (0.95w/v NaCl) and Euthatal (sodium pentobarbitone) were obtained from Fresenius Ltd. and the Veterinary Drug Company respectively. Lipopolysaccharide (from Pseudomonas aeruginosa) was obtained from Sigma.

The tobacco smoke was generated using 1R1 cigarettes purchased from the Institute of Tobacco Research, University of Kentucky, USA.

### Animals

Female inbred AJ mice (body weights on initial day of use: 17.2-27.4 g) were obtained from Harlan, full barrier bred and certified free from specified micro-organisms on receipt. The mice were housed, up to 5 per cage, in individually ventilated, polycarbonate solid bottomed cages (IVC) with grade 8 aspen chip bedding. Environment (airflow, temperature and humidity) within the cages was controlled by the IVC system (Techniplast). Food (RM 1, Special Diet Services) and water were provided ad libitum. Individual animals were identified by unique coloured “pentel” markings on their tails, weighed and randomly assigned to treatment groups.

### 2. Formulation

The required quantity of compound was placed into a mortar. Half the required volume CMC was slowly added to form a fine paste and then this was carefully added back to a container. The residual volume of CMC required to achieve the required dose is used to wash out the mortar and the washings added back to the container. For the combination dose, each compound was formulated at double the final required concentration and an equal volume of each compound added together.

Frequency of formulation: Compounds were formulated fresh every day prior to each p.o. dosing. Vehicle (0.5 methyl cellulose in water) was freshly formulated every 3 days and stored in aliquots at 4° C. These aliquots were bought up to room temperature prior to formulation of compounds.

### 3.0 Methods

Previous studies have established that the total numbers of cells recovered in the BAL are significantly elevated 24 h following the final TS exposure of 11 consecutive daily TS exposures, this time point was used in the study reported here. Previous studies have shown that peak BAL neutrophilia is achieved 24 h post intranasal challenge with LPS at 0.3 μg. In the studies reported here this dose of LPS and this time point were employed. Control animals received phosphate buffered saline (PBS) intranasally.

Protocols for the exposure of mice to TS or LPS, obtaining bronchoalveolar lavage (BAL), preparation of cytospin slides for differential cell counts are as outlined below.

### Exposure of Animals to TS Daily for 11 Consecutive Days

In this exposure protocol, mice were exposed in groups of 5 in individual clear polycarbonate chambers (27 cm×16 cm×12 cm). The TS from the cigarettes was allowed to enter the exposure chambers at a flow rate of 100 ml/min. In order to minimise any potential problems caused by repeated exposure to a high level of TS (6 cigarettes), the exposure of the mice to TS was increased gradually over the exposure period to a maximum of 6 cigarettes. The exposure schedule used in this study was as follows:

| | |
|---|---|
| Day 1: 2 cigarettes | (approximately 16 min exposure) |
| Day 2: 3 cigarettes | (approximately 24 min exposure) |
| Day 3: 4 cigarettes | (approximately 32 min exposure) |
| Day 4: 5 cigarettes | (approximately 40 min exposure) |
| Day 5 to 11: 6 cigarettes | (approximately 48 min exposure) |

A further group of mice were exposed to air on a daily basis for equivalent lengths of time as controls (no TS exposure).

### LPS Challenge

Approximately 3 min prior to intra-nasal challenge anaesthesia was induced by isofluorane inhalation. Vehicle (PBS) or LPS was instilled at 50 μl per mouse. The LPS concentration was 6 μg/ml (0.3 μg per mouse). Animals were allowed to recover in a heated box at 37° C. and then returned to the home cage.

### Bronchoalveolar Lavage and Cytospin Analysis

Bronchoalveolar lavage was performed as follows:

The trachea was cannulated using a Portex nylon intravenous cannula (pink luer fitting) shortened to approximately 8 mm. Phosphate buffered saline (PBS) containing heparin (10 units/ml) was used as the lavage fluid. A volume of 0.4 ml was gently instilled and withdrawn 3 times using a 1 ml syringe and then placed in an Eppendorf tube and kept on ice prior to subsequent determinations.

### Cell Counts:

Lavage fluid was separated from cells by centrifugation and the supernatant decanted and frozen for subsequent analysis. The cell pellet was re-suspended in a known volume of PBS and total cell numbers calculated by counting a stained (Turks stain) aliquot under a microscope using a haemocytometer.

Differential Cell Counts were Performed as Follows:

The residual cell pellet was diluted to approximately 10⁵ cells per ml. A volume of 500 μl was placed in the funnel of a cytospin slide and centrifuged for 8 min at 800 rpm. The slide was air dried and stained using ‘Kwik-Diff’ solutions (Shandon) as per the proprietary instructions. When dried and cover-slipped, differential cells were counted using light microscopy. Up to 400 cells were counted by un biased operator using light microscopy. Cells were differentiated using standard morphometric techniques.

Pharmacokinetic Evaluation of Plasma Levels of Theoplhylline after Oral Dosing in A/J Mice

Animals were weighed and marked and given theophylline (5 ml/kg) p.o. at either 3, 1 or 0.3 mg/kg. At specified intervals (15, 30, 60 or 240 minutes) following oral dosing with theophylline animals were terminally anaesthetised and blood collected by cardiac puncture into syringes containing 20U lithium Heparin in 5 ul. The collected blood was mixed and decanted into eppendorf tubes before centrifugation in a microftige. Plasma was collected and stored at −80° C. prior to analysis by an HPLC/MS/MS method. The equipment used in the measurement of plasma levels were a Micromann Quatro Micro Mass Spectrometer (Micromass UK Limited) and a Waters 2795 Alliance HT liquid chromatograph (Waters USA).

Six reference standard concentrations were prepared by spiking mouse plasma with stock concentrations of theophylline dissolved in methanol. The final concentrations of theophylline in mouse plasma were from 0.1 to 6 mg/l. Samples were prepared for analysis by adding 200 μl of acetonitrile (containing 0.25 mg/l dextrorphan as an internal standard) to 50 μl of each thawed sample and standard and mixed vigorously. Each sample and standard was then centrifuged at 1000 g for 2 minutes and the supernatant removed for LC-MS/MS analysis.

Analysis of theophylline and dextrorphan was carried out using reverse phase HPLC with tandem mass spectrometric detection (LC-MS/MS). Positive ions for parent compound and a specific fragment product were monitored in a Multiple Reaction Monitoring mode using a Micromass Quatro Micro Mass Spectrometer with Micromass MassLynx software version 4.0. A 25 μl aliquot of each sample and standard was injected onto the liquid chromatography system.

### 3.1 Treatment Regimes

In the TS study animals received vehicle (1% carboxymethyl cellulose), a PDE4 inhibitor (3 mg/kg), theophylline (0.3 mg/kg), dexamethasone (0.3 mg/kg) or a theophylline/dexamethasone combination (at 3 and 0.3 mg/kg respectively) orally at 1 hour prior to and 6 hours post tobacco smoke exposure (−1 h and +6 h) on each of the 11 days. In addition, animals receiving steroid or the steroid combination were dosed with steroid 20 h prior to the first TS exposure. The control group of mice (shams) received vehicle on days 1 to 11 and were exposed to air daily for a maximum of 50 minutes per day. BAL was performed on day 12, 24 h following the eleventh and final TS exposure.

In the LPS study animals were given vehicle (1% carboxymethyl cellulose), dexamethasone (0.3 mg/kg), theophylline (0.3 mg/kg) orally 20 and 1 hour prior to i.n. instillation of LPS and 6 hours post.

In the PK study mice were given theophylline only at 3, 1 or 0.3 mg/kg and animals were sacrificed and plasma samples taken 15, 30, 60 or 240 minutes later.

### 3.2 Data Measurement and Statistical Analysis

All results are presented as individual data points for each animal and the mean value was calculated for each group.

Since tests for normality were positive the data was subjected to a one way analysis of variance test (ANOVA), followed by a Bonferroni correction for multiple comparisons in order to test for significance between treatment groups. A “p” value of <0.05 was considered to be statistically significant. Percentage inhibitions were automatically calculated within the Excel spreadsheets for the cell data using the formula below:

$$\% Inhibition = 1 - ( \frac{\begin{matrix}Treatment group result - \\ sham group result\end{matrix}}{\begin{matrix}TS vehicle group result - \\ sham group result\end{matrix}} ) \times 100$$

Inhibition data for other parameters were calculated manually using the above formula.

### 4.0 Results

### 4.1 Inflammatory Response in the Bronchoalveolar Lavage Induced by Eleven Daily Consecutive Exposures to Ts (24 h Post Final Exposure)

In this study exposure to TS for 111 consecutive days induced an inflammatory response 24 h following the final exposure. This consisted of significant increases in BAL of neutrophils, macrophages, eosinophils, lymphocytes and epithelial cells in the bronchoalveolar lavage (BAL recovered from BAL fluid when compared with air exposed (sham) animals (all P<0.01). The increases in macrophages, neutrophils, eosinophils and lymphocytes indicate cell influx while the increase in BAL epithelial cells is probably indicative of reduced attachment of these cells.

### 4.2 Effect of Theophylline, Dexamethasone and a Theophylline/Dexamethasone Combination on the Inflammatory Response Induced in the Bronchoalveolar Lavage by Eleven Daily Consecutive Exposures to Ts (24 h Post Final Exposure)

Groups of mice were treated orally at 1 h prior to and 6 h post each of the 11 days of exposure with either vehicle or PDE4 inhibitor, theophylline, dexamethasone and one of 2 theophylline/dexamethasone combinations (theophylline 3 mg/kg+dexamethasone 0.3 mg/kg twice daily or theophylline 1 mg/kg+dexamethasone 0.3 mg/kg twice daily). Animals were sacrificed 24 h post the final exposure to TS/air. A BAL was performed and the total number of cells recovered counted. Data are presented as individual points and the mean values shown. Data are from 9-10 animals per group. Statistical analysis was by ANOVA. A “p” value of <0.05 was considered statistically significant. ns=not statistically significant.

Neither theophylline (3 mg/kg) or dexamethasone (0.3 mg/kg) when given orally every day for 11 days, 1 h prior to and 6 h post TS exposure, significantly inhibited the total number of cells recovered in the BAL. No statistically significant inhibitory effect was seen on any of the specific cell types.

In contrast, the combination of theophylline (3 mg/kg)/dexamethasone (0.3 mg/kg) when given orally every day for 11 days, 1 h prior to and 6 h post TS exposure, significantly inhibited the total number of cells recovered in the BAL by 63% (p<0.001). This effect on total cells was comprised of a 77%, 60% and a 66% inhibition of macrophages, epithelial cells and neutrophils respectively (all p<0.05). No statistically significant inhibition of lymphocytes or eosinophils was seen at either dose.

The combination of theophylline at the lower dose and dexamethasone (1 and 0.3 mg/kg, respectively) when given orally every day for II days, 1 h prior to and 6 h post TS exposure, significantly inhibited the total number of cells recovered in the BAL by 47% (p<0.001). This comprised of a 59% and a 66% inhibition of macrophages, epithelial cells and neutrophils respectively (all p<0.05). No statistically significant inhibition of epithelial cells, lymphocytes or eosinophils was seen.

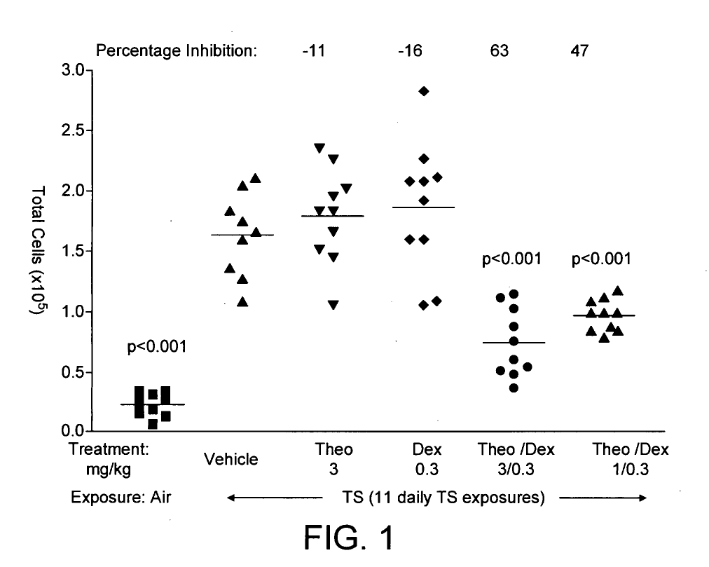

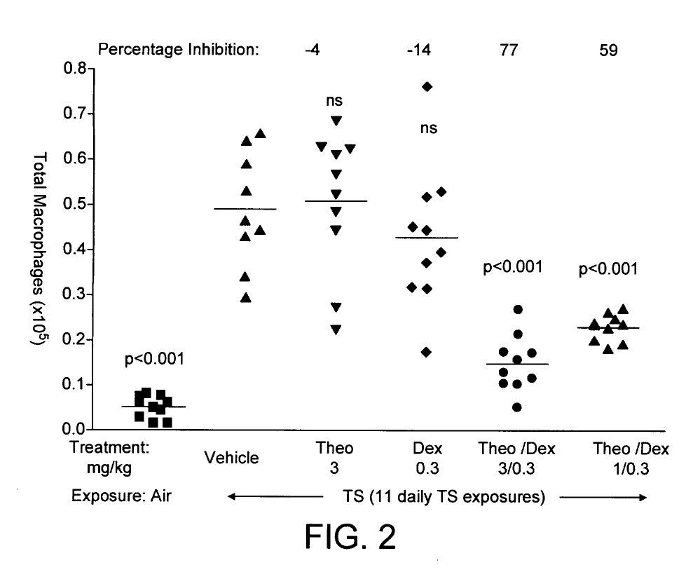

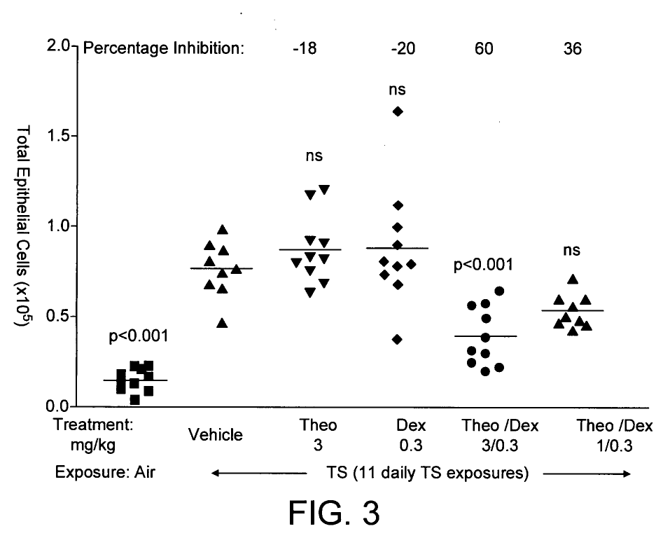

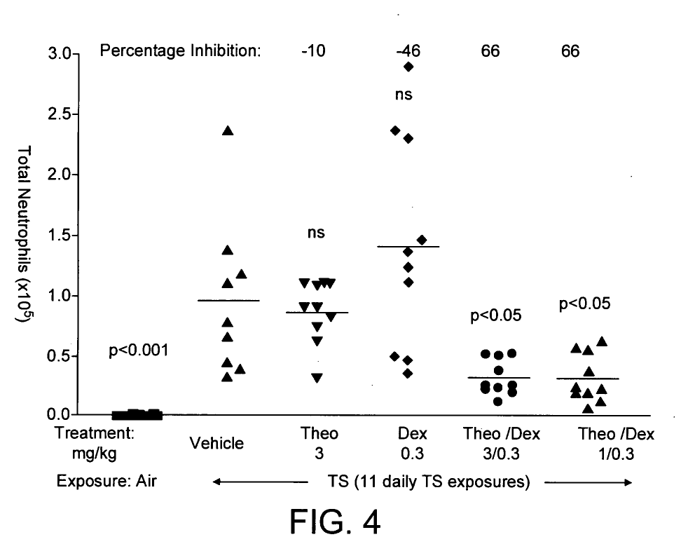

Degree and significance of inhibition is summarised in Table 1 and individual data is shown in FIGS. 1-4.

### 4.3 Effect of Theophylline and Dexamethasone on the Inflammatory Response Induced in the Bronchoalveolar Lavage by a Single LPS Challenge (24 h Post Challenge)

Mice were treated orally at 20 and 2 h prior to and 6 h post LPS challenge (0.3 μg) with vehicle, theophylline or dexamethasone. Animals were sacrificed 24 h post the LPS challenge. A BAL was performed and the total number of cells recovered counted. Data are presented as individual points and the mean values shown. Data are from 9-10 animals per group. Statistical analysis was by ANOVA. A “p” value of <0.05 was considered statistically significant. ns=not statistically significant.

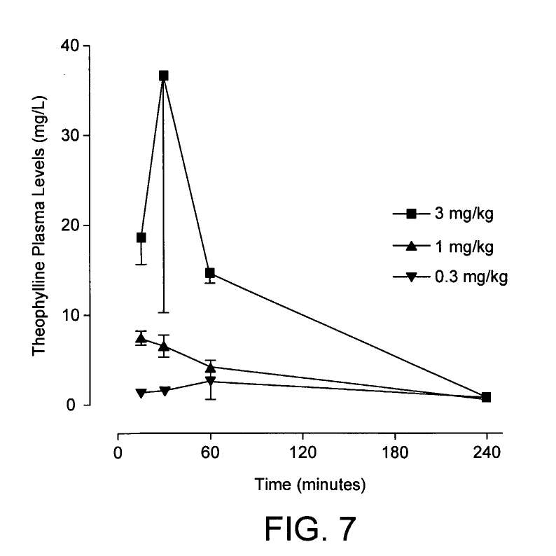

Intranasal administration of LPS to A/J mice resulted in an increase in the total number of cells recovered in the BAL 24 h following challenge (p<0.01). This increase in cells was comprised entirely of neutrophils. Dexamethasone when given orally (0.3 mg/kg) at −20, −1 and 6 h following LPS challenge significantly reduced total cells (68%, p<0.01) and neutrophils (71%, p<0.001) in BAL. Individual results are shown in FIGS. 7 and 8.

### 4.1 4.4 Phammacokinetic Analysis

Following oral dosing of theophylline to A/J mice plasma levels were detected at all doses administered. Plasma levels following oral administration of theophylline peaked at between 15 and 60 minutes with levels declining thereafter. Peak levels of theophylline were observed 30 minutes after administration of the 3 mg/kg dose (3.66±2.64 mg/l). At this dose plasma levels of drug remained below 5 mg/l for all animals at all time points bar one at the 30 minute time point (6.7 mg/A). Plasma concentrations at the 0.3 and 1 mg/kg dose were less than 1 mg/l at all time points analysed. Data is summarised in Table 2 and FIG. 7.

### 5.0 Discussion

In this study daily treatment with a steroid failed to have any inhibitory activity in this pulmonary inflammation model of COPD. This is in contrast to data obtained in other models, including the LPS model reported here. Theophylline also failed to demonstrate any significant anti-inflammatory activity in this COPD model.

However, when the compounds were co-administered at the same doses as given alone significant anti-inflammatory activity was seen (theophylline 3 mg/kg/dexamethasone 0.3 mg/kg). In a PK study plasma levels for theophylline given at a dose of 3 mg/kg were lower than 5 mg/l at all time points assessed which suggests that the synergistic effect of theophylline in the COPD model is achieved at plasma levels lower than those regarded as being necessary for anti-inflammatory efficacy (10-20 mg/l). Despite excellent inhibitory activity (>60%) of the combination of theophylline and dexamethasone (3 and 0.3 mg/kg) versus TS-induced increases in macrophages, epithelial cells and neutrophils no statistically significant inhibitory effect on increases in eosinophils and lymphocytes was seen. The level of inhibition seen with the combination suggests a truly synergistic effect has been uncovered by dosing the compounds together. This is further re-enforced by the efficacy seen at the lower dose combination of theophylline (1 mg/kg) with dexamethasone (0.3 mg/kg), this combination also had significant inhibitory activity on TS induced increases of macrophages (59%) and neutrophils (66%). Plasma levels of theophylline at 1 mg/kg remained below 1 mg/l at all time points assessed again suggesting that plasma levels lower than those normally required for efficacy are able to uncover steroid activity.

Intranasal administration of LPS to A/J mice on a single occasion resulted in a pulmonary neutrophilia 24 h following challenge. In contrast to the sub chronic TS model treatment with dexamethasone significantly reduced the LPS induced pulmonary inflammation demonstrating the steroid sensitivity of this model. Theophylline was without activity at the dose level tested.

These data demonstrate the steroid insensitivity of the mouse sub chronic TS model and furthermore reinforce the synergistic effect of combining a steroid at a therapeutic dose with an inactive dose of theophylline as a treatment paradigm for COPD. Critically, this effect is achieved at plasma levels lower than those normally associated with anti-inflammatory activity.

TABLE 1
Summary of the effects of theophylline, dexamethasone and a
theophylline/dexamethasone combination on TS induced inflammation
seen 24 h following 11 daily consecutive exposures

| Compound | Thophylline | | Dexamethasone | | Combination (T/D) | | Combination (T/D) | |
|---|---|---|---|---|---|---|---|---|
| Dose p.o. | 3 mg/kg | | 0.3 mg/kg | | 3/0.3 mg/kg | | 1/0.3 mg/kg | |
| twice daily | | | | | | | | |
| (−1 h and +6 h) | | | | | | | | |
| Inflammatory | Inhibition | | Inhibition | | Inhibition | | Inhibition | |
| markers (BAL) | | | | | | | | |
| Total Cells | −11% | n.s. | −16% | n.s. | 63% | p < 0.001 | 47% | p < 0.001 |
| Macrophages | −4% | n.s. | −14% | n.s. | 77% | p < 0.001 | 59% | p < 0.001 |
| Epithelial Cells | −18% | n.s. | −20% | n.s. | 60% | p < 0.001 | 36% | n.s. |
| Neutrophils | −10% | n.s. | −46% | n.s. | 66% | p < 0.05 | 66% | p < 0.05 |
| Eosinophils | −56% | n.s. | −94% | p < 0.01 | 7% | n.s. | 12% | n.s. |
| Lymphocytes | 1% | n.s. | −7% | n.s. | 40% | n.s. | 66% | n.s. |

n.s. not significant
+ signifies potentiation
Since tests for normality were positive the data was subjected to a one way analysis of variance test (ANOVA), followed by a Bonferroni correction for multiple comparisons, in order to test for significance between treatment groups. A “p” value of <0.05 was considered to be statistically significant.

TABLE 2
Plasma levels (mg/l) of theophylline after oral dosing in A/J mice

| Time post | Theophylline | | |
|---|---|---|---|
| oral dosing | Oral Dose (mg/kg) | | |
| (minutes) | 3 | 1 | 0.3 |
| | Plasma Levels (mg/l) | | |
| 15 | 1.86 ± 0.29 | 0.74 ± 0.76 | 0.13 ± 0.006 |
| 30 | 3.66 ± 2.64 | 0.66 ± 0.122 | 0.14 ± 0.025 |
| 60 | 1.47 ± 0.11 | 0.41 ± 0.084 | 0.256 ± 0.198 |
| 240 | 0.09 ± 0.1 | 0.06 ± 0.015 | 0.065 ± 0.011 |

data is summarised as the mean ± standard deviation

1. A method of treating a chronic respiratory disease in a subject in need thereof, comprising the administration of a methylxanthine compound and a steroid, wherein the methylxanthine compound is administered at a dose which, in isolation, is not effective in treating said respiratory disease.

2. The method of claim 1, wherein the steroid is administered at a dose which, in isolation, is not effective in reducing the inflammation associated with the respiratory disease.

3. The method of claim 2, wherein the steroid is administered at a dose which, in isolation, has minimal efficacy with respect to improvements in lung function and inflammation in treating said respiratory disease.

4. The method of claim 1, wherein the methylxanthine compound and the steroid act synergistically to treat inflammation in said respiratory disease.

5. The method of claim 1, wherein the methylxanthine compound and/or the steroid are administered by a route selected from the group consisting of inhalation, injection, oral administration and by means of long-term releasing implants.

6. The method of claim 4, wherein the methylxanthine compound and the steroid are administered by the same route.

7. A pharmaceutical composition in unit dosage form, comprising a methylxanthine compound at a dose which is insufficient to be effective in treating a respiratory disease if administered to a subject independently, and a steroid.

8. A pharmaceutical composition according to claim 7, wherein the steroid is provided at a dose which is insufficient to demonstrate anti-inflammatory activity in treating a respiratory disease if administered independently.

9. A pharmaceutical composition according to claim 7, wherein the treatment is the reduction of inflammation in a respiratory disease whereby the anti-inflammatory effect may result in an improvement in health status of the patient.

10. A kit for the treatment of a respiratory disease, comprising a methylxanthine compound and a steroid in unit dosage form, wherein the methylxanthine compound is provided at a dose which is insufficient to be effective in treating a respiratory disease if administered independently.

11. The kit according to claim 10, wherein the steroid is provided at a dose which is insufficient to demonstrate anti-inflammatory activity in treating a respiratory disease if administered independently.

12. A methylxanthine compound and a steroid in unit dosage form, wherein the methylxanthine compound is provided at a dose which is insufficient to be effective in treating a respiratory disease if administered independently, for simultaneous, simultaneous separate or sequential use in the treatment of a respiratory disease.

13. The methylxanthine compound and a steroid in unit dosage form of claim 12, wherein the methylxanthine compound and the steroid are provided at doses which are insufficient to demonstrate anti-inflammatory activity in treating a respiratory disease if administered independently, for simultaneous, simultaneous separate or sequential use in the treatment of a respiratory disease.

14. The method of claim 1, wherein the dose of methylxanthine compound used achieves plasma levels that are lower than that required for clinical efficacy (<5 mg/L).

15. The method of claim 1, wherein the dose of methylxanthine compound is administered by inhalation and is less than those required for clinical efficacy (30 mg to 500 mg).

16. The method of claim 1, wherein the dose of steroid is one that is used clinically which is either sub optimal or fails to demonstrate anti-inflammatory activity.

17. The method of claim 16, wherein the steroid is budesonide, either given alone or in combination with bronchodilators, and is administered twice daily at a dose of 400 μg or 800 μg respectively.

18. The method of claim 1, wherein the treatment of the respiratory disease is assessed by counting cells in or by bronchoalveolar lavage, induced sputum or bronchial biopsies.

19. The method of claim 18, wherein the cells are selected from the group consisting of macrophages, epithelial cells, neutrophils, eosinophils and lymphocytes.

20. The method of claim 18, wherein the cell count is reduced by 50% or more upon administration of a methylxanthine compound and a steroid.

21. The method of claim 20, wherein the cell count is reduced by 70% or more upon administration of a methylxanthine compound and a steroid.

22. The pharmaceutical composition of claim 7, wherein the amount of methylxanthine compound administered to the subject produces plasma levels that are insufficient for alleviating symptoms associated with a chronic respiratory disease.

23. The pharmaceutical composition of claim 22, wherein the methylxanthine compound plasma levels are less than 5 mg/L.

24. The pharmaceutical composition of claim 7, wherein the composition is formulated for administration by inhalation.

25. The pharmaceutical composition of claim 24, wherein the amount of methylxanthine compound administered by inhalation to the subject is between 30 mg to 500 mg.

26. The pharmaceutical composition of claim 7, wherein the steroid is present in an amount that is either sub-optimal or insufficient to alleviate inflammatory activity when administered to the subject.

27. The pharmaceutical composition of claim 7, wherein the steroid is budesonide.

28. The pharmaceutical composition of claim 27, wherein the amount of budesonide is 400 μg.

29. The pharmaceutical of claim 27, wherein budesonide is administered in combination with one or more bronchodilators.
